(19) United States
(12) Patent Application Publication
(10) Pub. No.: US 2020/0384219 A1
(43) Pub. Date: Dec. 10, 2020

(54) AUTOMATIC DOSING OF MEDICAMENTS IN ATOMIZERS

(71) Applicant: NEBU-TEC MED. PRODUKTE EIKE KERN GMBH, Elsenfeld (DE)

(72) Inventor: Stefan Kern, Elsenfeld (DE)

(21) Appl. No.: 16/758,633

(22) PCT Filed: Oct. 19, 2018
(86) PCT No.: PCT/DE2018/100859

§ 371 Date: Apr. 23, 2020

(30) Foreign Application Priority Data
Oct. 23, 2017 (DE) 10 2017 124 742.4

(51) Int. Cl. A61M 15/00; A61M 11/02

(52) U.S. Cl. CPC A61M 15/0066; A61M 15/0003; A61M 11/02; A61M 15/0025; A61M 15/0006

(57) ABSTRACT

A nebulizer unit for use in a nebulizer includes an aerosol generator, a housing, a medication reservoir and a cap. The aerosol generator having a supply side and a discharge side, which is retained between a sealing ring at the supply side and a retaining structure at the discharge side. The housing encloses the aerosol generator, the sealing ring and the retaining structure. The medication reservoir is integrated into the housing and includes an interior hollow volume, into which the medication liquid is introduced. The cap seals the reservoir, and includes a closure pin that subdivides the interior space of the reservoir into a first partial volume another partial volume. The liquid-tight closure being formed by the complementary partial surfaces. At least one of the two complementary partial surfaces of the closure pin and of the inner space of the reservoir having a rubber or silicone covering.

### CROSS-REFERENCE TO RELATED APPLICATION

This application is a Section 371 National Stage Application of International Application No. PCT/DE2018/100859, filed Oct. 19, 2018 the content of which is incorporated herein by reference in its entirety, and published as WO 2019/080964 A1 on May 2, 2019 not in English.

### FIELD

The present invention relates to a nebulizer unit having a medication reservoir for use in a nebulizer device, comprising an aerosol generator for atomizing a medication liquid having a supply side, which is in contact with the medication liquid present there, and a discharge side, at which the mist formed is emitted and is retained between a sealing ring at the supply side and a retaining structure at the discharge side, a housing, which encloses the aerosol generator, sealing ring and retaining structure and a medication reservoir, which is integrated into the housing at the supply side of the membrane and which can be closed by a tightly closing cap.

### BACKGROUND

In order to treat respiratory tract and lung diseases, an effective medication must be brought as completely as possible as far as the location of the disease, that is to say into the bronchial tubes, alveolar ducts or else the air sacs. For this purpose, a medication is usually prepared as a solution and atomized by means of a nebulizer before it is inhaled by the patient.

Nebulizers currently in use, which are also available as mobile devices, consist of four essential components:

the heart of the nebulizer is the so-called nebulizer unit, which contains a microperforated membrane, which by means of an oscillation generator, usually a piezo crystal, is set into rapid oscillation, usually in the ultrasonic range, by means of which the medication solution lying directly against the membrane is forced into the micrometre-sized holes of the membrane and is emitted at the discharge side of the membrane as a mist of fine droplets.

Usually, the membrane is mechanically coupled at its circumference directly or indirectly via a carrier plate to the circular oscillation generator to form an integrated unit, the aerosol generator. Either a tube with mouthpiece or a mouthpiece directly is mounted on the nebulizer unit. Furthermore, a nebulizer comprises a control unit, which generates the signals necessary to actuate the oscillation generator. At the supply side of the membrane, integrated into the nebulizer unit, there is usually located a reservoir for the medication solution, which provides an adequately dimensioned cavity, which can be firmly closed by means of a cap.

The reservoir must be adequately dimensioned so that it can receive different amounts of medication. With each inhalation, depending on the nature and severity of the disease and on the medication, a different dose of medication is necessary, which should usually be very precisely followed. The problem therefore arises of how this precise dosage can be ensured. This could be done first by metering out a dose required for inhalation from a larger container, and then filling this into the medication reservoir. However, this leads to the difficulty that, when used by persons who have not been appropriately trained or are not appropriately experienced, this dosage of extremely small amounts is very erroneous and imprecise, or the required aids are not available in the first place.

A further possibility provides for the dosing to be controlled electronically by the fact that that the control unit monitors the amount of liquid output by the aerosol generator. This is in principle possible in that the electrical and/or mechanical properties of the oscillation generator, such as the power consumed or the resonance frequency are dependent on the height of the particular liquid level. Thus, where the shape of the medication reservoir is known, the particular pressure can be determined by monitoring the current and voltage, and from the comparison of the current pressure with the pressure found at inhalation begin, the amount of liquid already discharged can be determined. However, this method is subject to relatively large uncertainties, so it is not suitable for really precise dosing.

The published application DE 10 2016 108 250 A2 presents a nebulizer unit with medication reservoir for use in a nebulizer, in which a cap of the medication reservoir has a displacer, which extends into the interior of the reservoir such that a residual volume corresponds to a medication dose to be administered.

The published patent application document US 2016/0193434 A1 discloses a dosage system for an inhalation device, in which a cylindrical displacer of a cap of a medication reservoir, when dipped into a filling chamber, a firmly defined medication dose is forced into a nebulizer chamber located below it, from where the medication is aerosolized. For the displacer, a comparatively flexible material, such as silicone, or, in so far as the filling chamber consists of a flexible material, for example silicone, a comparatively hard material is proposed.

The German utility model document DE 20 2015 102 538 U1 proposes fastening an oscillation generator and a support unit of an atomizer by means of a silicone adhesion promoter on a housing of the atomizer and covering them with a silicone cushion.

The German publication of unexamined application DE 10 2015 101 091 A1 discloses a piezoelectric nebulizer module with a double air chamber and improved sealing with respect to a simple sealing element of rubber or another elastomer.

In the German utility model DE 20 2011 110 785 U1, an aerosol generator is disclosed in which an oscillation generator is retained at both sides by sealing rings.

The document US 2017/0232211 A1 relates to the liquid reservoir for an aerosol generator with a liquid chamber and an opening in order to direct a liquid outward from the chamber and a cap for sealing the chamber. In some embodiments, the cap has a cylindrical connecting element with a collar at one lower end, the collar separating a partial volume of the chamber, which communicates with the opening, from a partial volume that is not in connection with the opening.

The published European application EP 1 205 198 A1 describes an aerosol medication dispensing device, which dispenses a metered dose of a medication in that the volume is controlled by a first chamber of a reservoir, the first chamber having a flexible partial surface.

The published application US 2003/0146300 A1 discloses a nebulizer with a nebulizing means and a metering chamber for a liquid medication and a second chamber for intercepting excess medication.

The application document US 2012/0216800 A1 discloses a medication dispensing apparatus having a reservoir for a liquid medication and a metering device, which is coupled thereto and has an upper and a lower chamber.

### SUMMARY

Against this background, it is the object of embodiments of the present invention to develop a nebulizer unit with medication reservoir, which enables a precise metering and provision of a medication to be used for inhalation, even by persons who are not experienced in this or who do not have the appropriate aids, and avoiding the wastage of potentially valuable medication liquid.

This object is achieved by means of a nebulizer unit with medication reservoir. Integrated into a closure cap of the reservoir is a closure pin, which subdivides the total volume of the reservoir into two partial volumes which are separate, that is two say which are not in fluid communication with one another, of which one is delimited at one side by the aerosol generator and has a volume that corresponds to a medication dose to be inhaled.

Despite the designation ‘closure pin’, the form of the body that is integrated into the cap, or mounted or fastened thereon, is, within the scope of embodiments of the invention, not necessarily pin-like or cylindrical. In the interests of minimizing the volume displaced by the pin, however, it is useful to have the slenderest possible centre section between an upper fastening on or in the cap and the surface portion of the closure pin which affects the closure.

In order to avoid the pin being undesirably, deformed, for example, undergoing bending during closing of the cap, in which, indeed, the cooperating partial surfaces of the pin and reservoir are pressed against one another, which always results in a certain axial compression of the pin, it is proposed to provide features that counteract this. This may be realized in the form of stiffening features, for example longitudinal ribs or longitudinal rails. Alternatively, or additionally, features serving to retain the form of the pin, such as rings or beads running transverse to the longitudinal direction can be provided.

The use of the nebulizer unit according to embodiments of the invention herein takes place such that first a medication is introduced from a transport container into the reservoir until at least the dose required for a single inhalation is present therein. Then the closure cap of the medication reservoir is put on and fastened, wherein, due to the closure pin, a partial volume adjacent to the aerosol generator is automatically separated from the rest of the reservoir, it thereby being ensured that the amount of medication that can be directly fed to the aerosol generator, that is to say the aerosolable amount of medication, corresponds exactly to the required dose.

Then, if appropriate, the control and the mouthpiece are connected to the nebulizer unit and inhalation is started. This can be continued until the control unit detects that the reservoir is completely empty.

The advantage of the nebulizer unit with automatic dosage according to embodiments of the present invention consists in the fact that, in a very simple manner, a rapid and precise metering of a required medication dose is permitted. A use requires neither aids for dosing, such as sterilized pipettes and transfer vessels, nor is it necessary to resort to an imprecise electronic measurement.

In order to meter out different medication doses, in the simplest embodiment of the present invention, closure caps with differently sized closure pins are provided. Since the caps are usually plastic parts that are relatively simple to manufacture, and which usually, with the inhalation of administered medication doses, comprise a discrete set of values, this is possible without too much outlay.

However, a more elegant solution consists in making the closure pin variable in its volume. This is possible, for example, in that the cap is of two-part construction: a circular part with a hole in the centre and a bent outer edge with which the connection to the medication reservoir is produced, as well as a cylindrical second part which is in the centre of the first part and serves as a closure pin.

By virtue of the screw connection, a user can set how far the closure pin projects into the reservoir interior and thus how much residual volume remains there. This is made easier for the user in that, on the outer side of the closure pin, which can be screwed in, a volume scale is registered, which indicates the residual volume that is still present. In order to achieve a completely (liquid-)tight closure, the screw thread may be provided with corresponding rubber seals or an elastic liquid-tight membrane is provided on the underside of the first part, which is stretched when the second part is screwed in, the volume that is present above this membrane and is separated from the interior of the membrane then forming the actual closure pin volume.

Since some medications that are applied by medication are very expensive substances, it is advantageous and economically appropriate to catch unrequired medication solution so that is available for subsequent use. This is possible thanks to the design of the medication reservoir and cap with closure pin which is presented here.

The use of the nebulizer unit according to embodiments of the invention with medication reservoir is thus for the application of a first inhalation dose as described above. The nebulizer unit according to the invention however additionally offers the possibility of reusing excess medication liquid.

Since, with filling of the medication into the reservoir and putting on of the caps, a separation between a first partial volume, delimited by the atomizer membrane or the aerosol generator, and a second volume comprising the rest of the medication reservoir is automatically ensured, unrequired medication being displaced into the latter. After completion of the first inhalation process, in a very simple manner, a second dose can be metered out, in that the cap is simply entirely or partially screwed off or taken off or rotated so that the form fit or areal fit of one side of the closure pin with the inner wall of the closure pin is eliminated and medication liquid can flow from the second partial volume into the first. Once this has taken place completely, the cover can be firmly put on and screwed again and a second inhalation process performed.

The advantage of the nebulizer unit according to embodiments of the invention is thus that medication liquid, which may possibly be very expensive, is not wasted but is available for further therapy.

In some aspects, the closure pin and the interior space of the reservoir are designed such that, when the cap is completely put on and closed, closure pin subdivides the interior space of the medication reservoir into at least two partial volumes: a first partial volume which is delimited at one side by the atomizer membrane or the aerosol generator and at the other side by an end surface or a partial surface of the closure pin, as well as a further, second partial volume, which comprises the rest of the reservoir.

In some embodiments, the closure pin, with the cap closed, bears against at least one circumferential line on the inner surface of the reservoir or comes at least very close thereto, so that an outer cross-section of the closure pin is (virtually) congruent with an inner cross-section of the reservoir.

This may be achieved for example in that the tip of the closure pin is designed as part of a rotational body, in particular as a cone or truncated cone, and the interior of the reservoir has a shape that is complementary to at least one part of this rotational body, in particular truncated cone, these two complementary parts conforming in a form-fitting manner with one another when closed.

In another embodiment, a portion of the reservoir is planar and an opening being present therein, of which the surface delimits a volume that is delimited at another side by the atomizer membrane. According to some embodiments of the invention, the closure pin is designed such that, when closed, a partial surface of the closure pin completely covers and closes the opening that is present in the planar surface. To ensure the leak-tightness of the closure, a seal, for example in the form of a sealing ring, may be provided at the closure pin side or at the reservoir side. Herein, neither the closure pin nor the stepped surface or opening may be rotationally symmetrical. For example, a surface may be provided such that it is not concentric with an axis of rotation of the cap and, after rotation of the cover, the opening is released or closed.

With the choice of the specific form of the complementary portion or partial surfaces of the closure pin and reservoir interior, however, it should be observed that the pressure inevitably generated on immersion of the closure pin into the reservoir, at least at conventional, axial immersion speeds, lies at all times below a pressure that can be maximally tolerated by the thin, and therefore very sensitive mesh membrane. Here, the time is particularly critical before production of the form fit between the closure pin (tip) and reservoir interior surface, when only a small gap remains through which the excess medication liquid can be displaced from the first partial volume into the second.

To circumvent this problem, embodiments of the present invention propose that the intermeshing complementary partial surfaces of the closure pin and of the reservoir are not in contact even when the cap is firmly closed, but a narrow gap remains between them. This is dimensioned such that, on one hand, it is large, that is to say wide, enough to enable problem-free displacement of the liquid without permitting excessively high pressure peaks, but on the other hand is small, that is to say narrow, enough to prevent flow of liquid from the second partial volume into the first, which empties in the course of inhalation. This is possible because the medication liquid possesses a finite surface tension, so that, as long as the gap is not too wide, a liquid residue remains therein, which closes the gap like a seal and effectively hinders the inflow of liquid.

It is also important here that the dimension of the contact surface in the axial direction is large enough, in any case orders of magnitude above the width of the gap, that is to say the distance between the complementary partial surfaces. Variations of this point-to-point distance are to be kept to a minimum, in particular it is important that the distance, that is to say the gap width, is not too large at any point. What would be “too large” here depends crucially on the specific form of the complementary surfaces, on the surface tension of the liquid and the minimally generated sub-atmospheric pressure in the first partial volume during atomization.

Advantageous embodiments of the present invention, which can be realized individually or in combination, in so far as they do not obviously preclude one another are described below.

In one embodiment, the closure pin is embodied essentially as a cylinder and, in order to save material and weight, is made hollow. Furthermore, to produce a tight form-fit with a correspondingly shaped part of the inner surface of the reservoir, it is advantageous if the tip of the closure pin has a conical or truncated conical shape. To ensure the subdivision of the inner volume of the reservoir into two partial volumes, a cross-section of the closure pin is brought into congruence with an inner cross-section of the reservoir. These two cross-sections do not need to be rotationally symmetrical, however in practice this is advisable to ensure a reliably tight closure.

To reduce the required manufacturing tolerances for a reliable closure, it is proposed to provide the intermeshing complementary partial surfaces of closure pins and/or reservoir inner surface with an elastic material. This may be realized in the form of a sealing ring inserted in the reservoir or placed around the tip of the closure pin. It would also be possible to provide one or both of the partial surfaces with a covering of an elastic material. In particular rubber or silicone would be suitable for this.

Alternatively the entire pin can also be made of a material that is elastic but not too soft. Silicone is particularly suitable for this. This design has the advantage of clearly increasing the necessary manufacturing tolerances that have to be maintained, roughly in the range of half a millimetre in case of nebulizers of customary size.

Particularly with this embodiment of the closure pin, it is recommended to provide stiffening and/or shape-retaining features. In one possible embodiment, these may be stiffening ribs running parallel to the longitudinal direction of the pin. In another embodiment, shape-retaining rings may alternatively or additionally run transversely to this direction. By these means the shaft of the pin is it is effectively prevented from being deformed by lateral bending such that the areal fit of the cooperating partial surfaces of the pin tip and medication is compromised.

Alternatively to the above-described separation of the two partial volumes by means of a form-fit between the outer surface of the closure pin and the inner surface of the reservoir, embodiments of the present invention propose to effect this by means of an areal fit between an end surface and/or a lateral surface of the closure pin and a stepped surface, that is to say an essentially planar surface with an opening therein, which leads to the aerosol generator. To ensure tight closure, it may be necessary to provide a seal either at the closure pin side or at the reservoir side, for example in the form of a flexible plastic covering or a sealing ring. The opening in the stepped surface may here be symmetrical to an axis of rotation of the closure pin or of the cap, or it is placed in a non-symmetrical position, such that, on rotation of a closure pin, which is also not symmetrically shaped, the opening can also be released when no axial displacement of the cap takes place.

This may be realized, for example, if closure of the cap is performed by means of a rotational closure, in which at the end of the rotational range the thread has a vanishing pitch, such that no further axial displacement takes place in this rotational range.

As further closure possibilities, apart from a rotational closure, a screw of bayonet closure can also be used. Here, to produce a secure closure, it is necessary that the cap and reservoir have a common axis, the rotational axis.

At the outlet side of the membrane or of the aerosol generator, either a mouthpiece should appropriately be formed or a connection means for such should be provided. This connection piece can be formed as a hollow cylinder with closed lateral surfaces.

It is further advisable to choose the total volume of the reservoir, with the deduction of the minimum closure pin volume, so large that it is larger than the normal volume in normal medication transport containers.

Further properties and features of embodiments of the present invention are described in greater detail below with reference to the figures of exemplary embodiments. These are only intended to illustrate the invention, and in no way to limit its generality.

### BRIEF DESCRIPTION OF THE DRAWINGS

Wherein:

FIG. 1 shows a longitudinal section through a basic version of a nebulizer unit according to the invention with two-part medication reservoir.

FIG. 2 shows a longitudinal section through an embodiment with two-part medication reservoir in which the size of the first partial volume can be varied.

FIG. 3 shows two longitudinal sections through an embodiment of the present invention in which an areal fit takes place.

FIG. 4 in two partial figures shows embodiments of the closure pin of the nebulizer according to the invention.

### DETAILED DESCRIPTION OF ILLUSTRATIVE EMBODIMENTS

FIG. 1 shows the longitudinal section through a preferred embodiment of the nebulizer unit according to the present invention, in which a two-part medication reservoir is present, the separation of the two partial volumes 110, 111 from one another taking place by means of a form fit between the tip of one of the cylindrical closure pins 131 and a region of the reservoir 11 that is of complementary shape.

The housing 10 can further be seen, which encloses the aerosol generator 101, which comprises the membrane, the sealing ring 102 and the retaining structure 103 and a mouthpiece connection at the membrane-output side, as well as a medication reservoir 11 at the membrane input side. The latter can be closed by means of a tightly closing cap 13, which is provided with the closure pin 131 according to the invention in the form of a cylinder with a truncated conical tip. When the cap 13 is tightly closed, as illustrated, a form fit between this truncated conical tip of the closure pin 131 and a region in the interior of the reservoir 11 is produced, so that the subdivision into the two partial volumes 110, 111 takes place. After completion of an inhalation, that is to say when the first partial volume 110, is completely emptied, a second inhalation with medication dosing that is also precise can take place simply by removing the cap 13 entirely or partially, so that the medication can flow from the second partial volume 111 into the first partial volume 110 and then the cap 13 can be tightly put on again.

FIG. 2 shows a longitudinal section through a different embodiment of the nebulizer unit of the present invention, in which a form fit between the tip of the closure pin 131 and a region of the medication reservoir 11 is also produced. In addition to the embodiment shown in FIG. 1, however, the cap 13 is designed in two parts. It thus consists of the actual cover 134 and a bolt 133, which can be screwed therein by screw thread, of which the tip can project to a more or less extent into the first partial volume 110, so that its remaining effective residual volume can be varied. As in the nebulizer unit shown in FIG. 1, a connection 12 for a mouthpiece is also provided here.

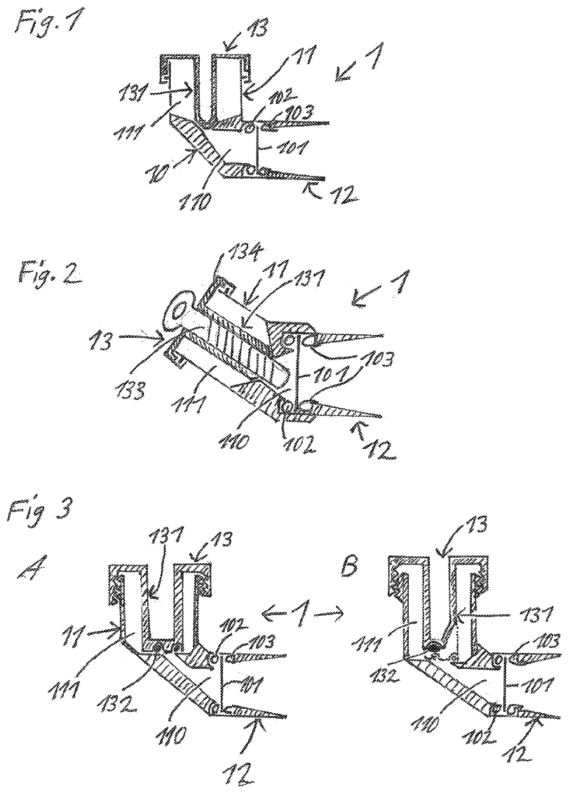

FIG. 3 shows, in two partial views, longitudinal sections through a third preferred embodiment of the present invention, in which not a form-fit but an areal fit takes place between an end surface of the closure pin 131 as well as a stepped surface in the interior of the reservoir 11, which is tightly sealed by means of a sealing ring 132 that is mounted at the closure pin side 132.

In the section shown in partial figure A, the cap 13 with closure pin 131 is firmly put on the medication reservoir 11 and sealed. By means of area connection between an end surface of the closure pin 131 with a stepped surface in the interior of the reservoir 11, a subdivision into two mutually separated partial volumes 110 and 111 is produced. The tightness of the areal fit is ensured by means of a sealing ring 132 fastened at the closure pin side.

Partial figure B shows the section from partial figure, however here the cover 13 is not firmly closed but rotated through approx. 90 degrees compared to the state shown in partial figure A, as well as displaced axially slightly upward due to the guidance of the cover rim in the screw thread of a screw closure. As can be seen, due to an asymmetric shape of the closure pin 131, as a cylinder with a bevelling at the lower end, it is achieved that, in this position, the opening in the stepped surface, which is adjacent to the partial volume 110, is released. The liquid-tight separation of the two partial volumes 110, 111 is thus eliminated; a liquid exchange can take place if required.

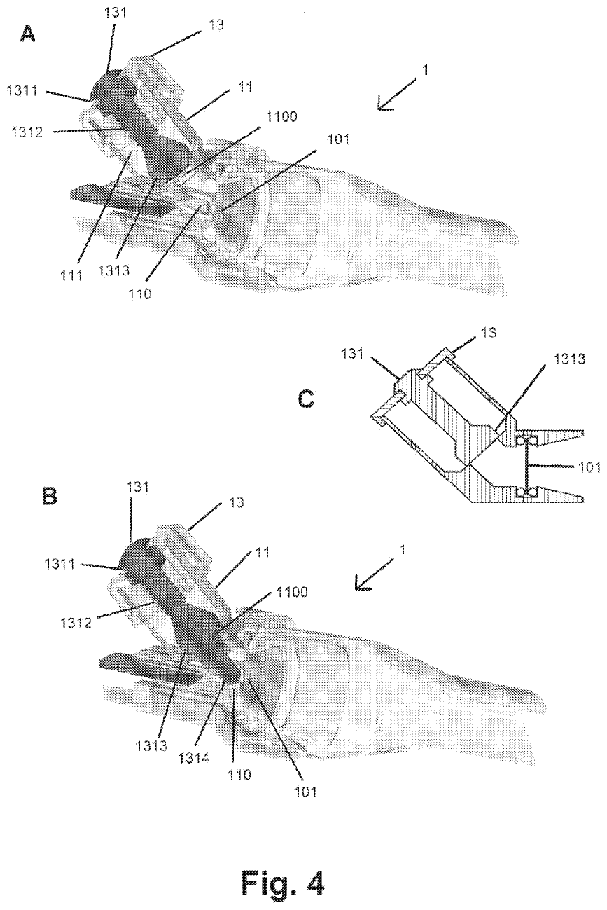

FIG. 4 shows possible embodiments of a one-piece solid closure pin according to the present invention.

The pin 131 from partial figure A is retained by means of a double collar 131 in an opening in cap 13. The shank of the pin has, as shaping features, a row of circumferential beads 1312, transverse to the longitudinal axis of the pin 1312. These have the effect that the pin is not so easily bent on axial compression. That part 1313 of the pin 131 that effects closure of opening 1100 and cooperates with the inner surface of reservoir 11 is designed as an upwardly tapering truncated cone. A different shape, for example with a curved instead of straight profile of the sides would also be conceivable.

In partial figure B, the pin 131 additionally has a displacer tip 1314, which starts at the base of the closure part 1313 and projects into the partial volume 110, and reduces the volume thereof corresponding to the volume of the tip By this means, smaller medication doses can be administered in comparison to the embodiment from partial figure A.

Partial figure C shows a schematic longitudinal section through the nebulizer unit from partial figure A.

### LIST OF REFERENCE CHARACTERS

1 Nebulizer unit
10 Housing
101 Aerosol generator
102 Sealing ring
103 Retaining structure
11 Reservoir
110 First partial volume
1100 Opening
111 Further partial volume
12 Connecting means for mouthpiece
13 Cap
131 Closure pin
1311 Double collar
1312 Shape-retaining ring
1313 Closure part
1314 Displacer tip
132 Sealing ring
133 Screw-in bolt
134 Cover
2 Mouthpiece

Although the embodiments of the present disclosure have been described with reference to preferred embodiments, workers skilled in the art will recognize that changes may be made in form and detail without departing from the spirit and scope of the present disclosure.

1-14. (canceled)

15. A nebulizer unit with a medication reservoir for use in a nebulizer, comprising:
an aerosol generator for atomizing a medication liquid having a supply side, which is in contact with the medication liquid present there, and a discharge side, at which a mist formed is emitted and which is retained between a sealing ring at the supply side and a retaining structure at the discharge side;
a housing, which encloses the aerosol generator, the sealing ring and the retaining structure;
the medication reservoir integrated into the housing at the supply side of the aerosol generator, the medication reservoir having an interior hollow volume, into which the medication liquid is introduced; and
a cap, which tightly seals the reservoir,
wherein:
the cap includes a closure pin, which extends into the interior space of the reservoir;
a portion of the surface of the closure pin and a portion of the inner surface of the reservoir being formed complementary to one another and, when the cap is firmly fitted, form a liquid-tight closure, by means of which the inner space of the reservoir is subdivided into a first partial volume, which is delimited at one side by the aerosol generator and on another side by an end surface or a partial surface of the closure pin, as well as at least one further partial volume;
the liquid-tight closure being formed by the complementary partial surfaces being close to one another, one on top of the other, or are pressed against one another; and
at least one of the two complementary partial surfaces of the closure pin and of the inner space of the reservoir having a rubber or silicone covering.

16. The nebulizer unit according to claim 15, wherein the complementary partial surfaces of the closure pin and of the reservoir are not in contact with one another even when the cap is firmly closed, but a narrow gap remains open between them.

17. The nebulizer unit according to claim 15, wherein the closure pin is cylindrical, and/or is hollow or solid.

18. The nebulizer unit according to claim 15, wherein the closure pin comprises a rotationally symmetrical, in particular a conical or truncated-conical lower end.

19. The nebulizer unit according to claim 15, wherein the inner space of the reservoir contains a section that is complementary to at least a portion of the tip of the closure pin, of which section the inner surface of the cap, when completely closed, conforms in a form-fitting manner circumferentially to the tip of the closure pin, and thus achieves an at least almost complete spatial separation of the first partial volume, which is closed off at one side by the aerosol generator, from a second partial volume.

20. The nebulizer unit according to claim 15, wherein in the interior space of the reservoir, an essentially planar stepped surface is present, which has an opening to a partial volume that is delimited at a different side from the aerosol generator, and the closure pin has an end surface that is larger than the opening in the stepped surface and covers and seals the latter when closed.

21. The nebulizer unit according claim 15, wherein the opening or the end surface of the closure pin is disposed relative to an axis of rotation of the cap, such that a rotation entirely or partly releases the opening.

22. The nebulizer unit according to claim 15, wherein the closure pin has a variable volume.

23. The nebulizer unit according to claim 22, wherein the volume of the closure pin is variable by means of a bolt that is guided in a thread in the cap.

24. A nebulizer unit with a medication reservoir for use in a nebulizer, comprising:
an aerosol generator for atomizing a medication liquid having a supply side, which is in contact with the medication liquid present there, and a discharge side, at which a mist formed is emitted and which is retained between a sealing ring at the supply side and a retaining structure at the discharge side,
a housing, which encloses the aerosol generator, the sealing ring and the retaining structure;
the medication reservoir being integrated into the housing at the supply side of the aerosol generator, the medication reservoir having an interior hollow volume, into which the medication liquid is introduced; and
a cap, which tightly seals the reservoir,
wherein:
the cap includes a closure pin, which extends into the interior space of the reservoir;
a portion of the surface of the closure pin and a portion of the inner surface of the reservoir being formed complementary to one another and, when the cap is firmly fitted, form a liquid-tight closure, by means of which the inner space of the reservoir is subdivided into a first partial volume, which is delimited at one side by the aerosol generator and on another side by an end face or a partial face of the closure pin, as well as at least one further partial volume;
the liquid-tight closure being formed by the complementary partial surfaces being close to one another, one on top of the other, or pressed against one another; and
the closure pin being made entirely of silicone and, as a stiffening feature, comprising stiffening ribs running in a longitudinal direction of the closure pin and/or as shape-retaining feature, comprising shape-retaining rings running transverse to the longitudinal direction.

25. The nebulizer unit according to claim 24, wherein the complementary partial surfaces of the closure pin and of the reservoir are not in contact with one another even when the cap is firmly closed, but a narrow gap remains open between them.

26. The nebulizer unit according to claim 24, wherein the closure pin is cylindrical, and/or is hollow or solid.

27. The nebulizer unit according to claim 24, wherein the closure pin comprises a rotationally symmetrical, in particular a conical or truncated-conical lower end.

28. The nebulizer unit according to claim 24, wherein the inner space of the reservoir contains a section that is complementary to at least a portion of the tip of the closure pin, of which section the inner surface of the cap, when completely closed, conforms in a form-fitting manner circumferentially to the tip of the closure pin, and thus achieves an at least almost complete spatial separation of the first partial volume, which is closed off at one side by the aerosol generator, from a second partial volume.

29. The nebulizer unit according to claim 24, wherein in the interior space of the reservoir, an essentially planar stepped surface is present, which has an opening to a partial volume that is delimited at a different side from the aerosol generator, and the closure pin has an end surface that is larger than the opening in the stepped surface and covers and seals the latter when closed.

30. The nebulizer unit according claim 24, wherein the opening or the end surface of the closure pin is disposed relative to an axis of rotation of the cap, such that a rotation entirely or partly releases the opening.

31. The nebulizer unit according to claim 24, wherein the closure pin has a variable volume.

32. The nebulizer unit according to claim 30, wherein the volume of the closure pin is variable by means of a bolt that is guided in a thread in the cap.

33. A method of using a nebulizer unit for use in a nebulizer, the nebulizer unit comprising:
an aerosol generator for atomizing a medication liquid having a supply side, which is in contact with the medication liquid present there, and a discharge side, at which a mist formed is emitted and which is retained between a sealing ring at the supply side and a retaining structure at the discharge side;
a housing, which encloses the aerosol generator, the sealing ring and the retaining structure;
the medication reservoir integrated into the housing at the supply side of the aerosol generator, the medication reservoir having an interior hollow volume, into which the medication liquid is introduced; and
a cap, which tightly seals the reservoir,
wherein:
the cap includes a closure pin, which extends into the interior space of the reservoir;
a portion of the surface of the closure pin and a portion of the inner surface of the reservoir being formed complementary to one another and, when the cap is firmly fitted, form a liquid-tight closure, by means of which the inner space of the reservoir is subdivided into a first partial volume, which is delimited at one side by the aerosol generator and on another side by an end surface or a partial surface of the closure pin, as well as at least one further partial volume;
the liquid-tight closure being formed by the complementary partial surfaces being close to one another, one on top of the other, or are pressed against one another; and
wherein:
at least one of the two complementary partial surfaces of the closure pin and of the inner space of the reservoir having a rubber or silicone covering; or
the closure pin being made entirely of silicone and, as a stiffening feature, comprising stiffening ribs running in a longitudinal direction of the closure pin and/or as shape-retaining feature, comprising shape-retaining rings running transverse to the longitudinal direction
the method comprising:
completely filling the reservoir with a medication solution from a transport container;
fastening the cap to the reservoir, wherein only a desired dose of medication is provided for inhalation;
connecting a mouthpiece and a control unit; and
inhaling until the medication solution is consumed.

34. The method according to claim 33, further comprising:
metering out a second medication dose by taking off or rotating the cap entirely or partially, wherein the medication liquid present in the second partial volume flows into the first partial volume until the latter is completely filled;
forming a separation of the two partial volumes using the cap; and
inhaling the medication liquid in the first partial volume.
